(19) United States
(12) Patent Application Publication
(10) Pub. No.: US 2025/0367362 A1
(43) Pub. Date: Dec. 4, 2025

(54) APPARATUS FOR DELIVERING LIQUID TO A SURFACE

(71) Applicant: Catalyst LLC, Clancy, MT (US)

(72) Inventor: Michael J. Dixon, Clancy, MT (US)

(21) Appl. No.: 18/878,052

(22) PCT Filed: Jul. 3, 2023
(86) PCT No.: PCT/US2023/069576

§ 371 Date: Dec. 22, 2024

Related U.S. Application Data
(60) Provisional application No. 63/358,079, filed on Jul. 1, 2022.

(51) Int. Cl. A61M 3/02

(52) U.S. Cl. CPC A61M 3/022; A61M 2206/20; A61M 2210/0618

(57) ABSTRACT

A device for delivering fluid to a surface said device comprising: a housing having a forwardmost end and a trailing end, the trailing end formed to be received by a container and be in fluid communication therewith; a hollow cavity defining the interior of said housing; and an interface disposed on the forwardmost end of said housing to be received by a surface; wherein said forwardmost end comprises a turbulating structure characterized by a plurality of holes having an orientation at an angle from square to said interface, and a fence perpendicular to said interface superjacent said turbulating structure.

### CROSS REFERENCE TO RELATED APPLICATIONS

This application claims priority to U.S. Application No. 63/358,079, filed on Jul. 1, 2022, the contents of which are hereby incorporated by reference in its entirety.

### TECHNICAL FIELD

This invention generally relates to a method and apparatus for delivering liquid by way of vortex hydrodynamics.

### BACKGROUND

Nasal lavage is an ancient practice that still has a fundamental role in the management of sinonasal conditions. Nasal lavage has evolved significantly since the first considered for medical use and has always played a central role in the treatment of patients. Further innovation is still necessary to address the shortcomings of current nasal lavage systems.

Nasal irrigation is typically used alone or in association with other therapies in several conditions including chronic rhinosinusitis and allergic rhinitis. However, despite it being largely prescribed in everyday clinical practices, current devices do not provide optimal delivery of liquid medication to patients.

This background information is provided for information purposes only. No admission is intended, nor should it be construed, that any of the preceding information constitutes prior art against the present invention. In addition, the preceding information should not be construed to mean that a search has been made or that no other pertinent information as defined in 37 CFR § 1.56(a) exist.

### SUMMARY

The present disclosure provides a method and apparatus for delivering liquid to a surface. The liquid may be applied for nasal and body cavity irrigation and lavage. The method and apparatus disclosed herein may be used to apply liquid to other surfaces such as a biological surface, an inanimate surface, etc. The method and apparatus disclosed herein may utilize a turbulent liquid characterized by a vortex. Without wishing to be bound by any particular theory, it is believed that the washing of a surface (e.g., a body cavity or the like) with turbulent vortex-like fluid streams may more effectively and thoroughly rinse and treat areas through sloshing of the fluid.

The inventor surprisingly discovered a method and apparatus for lavage and irrigation of body cavities, such as the nasal, paranasal sinus or the like by way of fluids having a vortex flow profile and having increased turbulence. Such a flow pattern may be obtained via an apparatus having a fluid container and a forward interface, the forward interface having a plurality of canted holes and a fence configured to turbulating a fluid flowing therethrough. At such time that a fluid passes through the interface, it may gain a combination of angular and axial momentum so that it may assume a “double helix” profile and may experience increased turbulence. Without wishing to be bound by any particular theory, it is believed that due to this increased turbulence, the body cavity is more gently and thoroughly rinsed.

One aspect of the invention pertains to a device for delivering fluid to a surface (e.g., nasal cavity and paranasal sinus) and rinse thereof, said device comprising:
a housing having a forwardmost end and a trailing end formed to be received by a fluid container and be in fluid communication therewith, a hollow cavity defining the interior of said housing, for forward discharging the fluid provided from the fluid container depending on pressures applied to said fluid; and an interface disposed on the forwardmost end of said housing to be received by the cavity further comprising a turbulating structure defined by a plurality of holes having an orientation at an angle from square to said interface, and a fence perpendicular to said interface superjacent to said turbulating structure;
wherein the fluid by way of being transported through said plurality of holes and interacting with said fence becomes turbulent and forms a vortex.

A further aspect of the invention pertains to a method for applying a fluid to a surface, the method comprising the steps of:
a. sealingly engaging an interface of a surface
b. pressurizing a fluid urging the fluid to enter, and while so entered, engaging with the apparatus;
c. a fluid injection step in which a turbulent vortex of fluid generated by said pressurized fluid engaging said apparatus is injected into a surface;
d. a rinsing step in which the surface is rinsed upon interaction with said vortex of fluid.

A yet further aspect of the invention pertains to a nozzle assembly comprising a housing of the type having a body that has an inlet thereto and an outlet therefrom; a core member and a nozzle structure the core member defining the interior of the nozzle and providing fluid communication between the inlet and the outlet; said nozzle structure having a plurality of holes about the face of said nozzle adapted for fluid transfer, and a fence structure for directing said fluid; wherein said holes said nozzle have a canted orientation relative to the forwardmost surface.

A yet further aspect of the invention pertains to a device comprising a housing having an orifice that opens into a chamber; and a cap sealing attached thereto; wherein the cap has a first end with a first opening, a second end with chamfered tip having a plurality of second openings disposed in an annular arrangement and having a fence for directing fluid flow, and a hollow channel extending from the first opening to the plurality of second openings; wherein the first end is attached to the hosing proximate the orifice; wherein the tip is shaped and dimensioned to be inserted partially into. And for a substantially fluid-resistant seal with, a surface; wherein each of the plurality of second opening is canted relative to the longitudinal axis and adjacent second opening such that, as liquid is first urged simultaneously thorough the plurality of second openings and second interacts with said fence, the liquid experiences a vortex flow pattern.

### BRIEF DESCRIPTION OF THE DRAWINGS

FIG. 1 is a cross sectional view of an exemplary embodiment of a device according to the present disclosure.

FIG. 2 is a top view of an exemplary embodiment of a turbulating structure according to the present disclosure.

FIG. 3 is a schematic of an exemplary embodiment of a turbulating nozzle according to the present disclosure.

FIG. 4 is a plan view of an exemplary embodiment of the invention according to the present disclosure.

### DETAILED DESCRIPTION

### Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “comprises,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

As used herein, the term “fluid” refers to a liquid, gas, or other material that continuously deforms under an applied shear stress, or external force.

As used herein, the term “rinse” refers to mechanically moving foreign matter by hydraulic interaction with a fluid.

As used herein, the term “laminar” refers to a fluid having fluid particles following smooth paths in layers, with each layer moving smoothly past the adjacent layers with little or no mixing.

As used herein, the term “turbulent” refers to fluid motion characterized by chaotic changes in pressure and flow velocity.

As used herein, the term “vortex” refers to a region of fluid flow in which the flow revolves around an axis line, which may be straight or curved. In some embodiments, a vacuum may be formed in the center proximal the axis of fluid rotation.

As used herein, the term “fence” refers to a rigid structure configured to direct and manipulate the flow of fluid in interaction therewith.

As used herein, the term “turbulating structure” refers to any structure that has at least one surface configure that, during interaction with a fluid, the surface urges the fluid to transition to turbulent flow.

As used herein, the term “body cavity” generally refers to, but is not limited to nasal cavity, paranasal sinuses, external auditory canal, nasopharynx, eustachian tube, oral cavity, oropharynx, hypopharynx, larynx, trachea, lung, esophagus, stomach, anus, rectum, large and small intestine, urethra, bladder, vagina, cervix, uterus, and eye socket.

As used herein, the term “surface” refers to the outermost or uppermost layer of an object. The term “surface” as used herein includes the outermost or uppermost layer of an object, said object comprising plastic and/or metal. The object may also be the body cavity or other area of a mammal body.

As used herein, the term “body surface” refers to a surface of skin or other biological tissue (such as intraperitoneal, face, scalp, etc.). In some embodiments, “body surface” refers to the nasal, paranasal, or sinus.

The present disclosure generally relates to improved methods and devices for nasal lavage and irrigation. The devices of the present disclosure utilize a novel spray head which are configured to increase the turbulence of fluid flow from a container (such as a bottle), and in some embodiments, form a vortex of fluid. Such turbulent or vortex fluid is more suitable for nasal lavage and irrigation. In some embodiments, the container comprises a bottle.

Advantageously, in some embodiments, the devices and methods of the present disclosure include features that provide a spray head adapted to increase the turbulence of liquid used for nasal lavage and irrigation of other body cavities. Moreover, the device and methods optionally employ advanced techniques for introducing medication to a surface.

In some aspects, the present disclosure provides a method for nasal and body cavity lavage and irrigation, said method comprising applying a liquid to a surface such as a body cavity or other body part using a device as described herein.

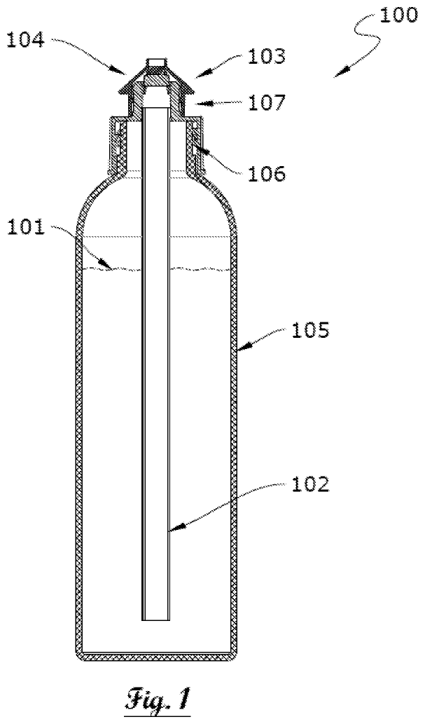

FIG. 1 shows a cross section of an exemplary embodiment of the invention involving a container and fluid delivery device 100 according to the present disclosure wherein said device is attached to a container. A method for nasal and body cavity lavage comprises applying a pressure to a fluid 101, the increased pressure urging the fluid to travel through a tube 102 to a fluid delivery device 103. At such time the forwardmost interface 104 of the fluid delivery device should be sealingly engaged with a body cavity. After such time that the fluid 101 becomes pressurized and urged to enter the fluid delivery device 103, the fluid will become turbulent and exhibit vortex-like fluid dynamics prior to entering a body cavity. Thereupon entering, and as a result from the increased turbulence, the body cavity is rinsed.

Continuing with FIG. 1, a fluid container 105 is shown. Such a fluid container may be a plastic bottle or the like. In other embodiments such a container may be a flexibly resilient container. In some embodiments, the fluid delivery device 103 is sealably attachable to a container 105 by way of sealing means 106. In some embodiments, the fluid delivery device 103 contains a means for switchably discontinuing fluid ejection 107. Such a feature is particularly useful for storage of fluid. Further, in some embodiments, it is envisioned the fluid 101 may be water, saline, or a medicated substance, e.g., one or more medications dissolved or suspended in a liquid.

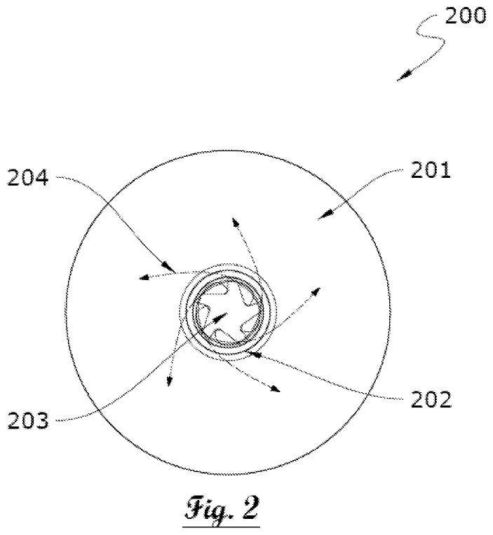

FIG. 2 shows a top-down view of an arrangement of a spray head 200 as described herein. The body cavity interface 201 (demonstrates an exemplary surface interface) may be configured to interact with a body cavity and form a substantially watertight seal therewith. In particular arrangements, the body cavity interface 201 is adapted to be received by the body cavity (e.g., nostril). In some embodiments, the body cavity is a nostril. A turbulating structure 203 may be included, which is adapted for imparting angular momentum onto a fluid stream. In some embodiments, a fence structure 202 is included and may be configured to impart a normal force onto a fluid stream. It is envisioned that the combination of such normal force and angular momentum will establish a turbulent vortex of fluid 204.

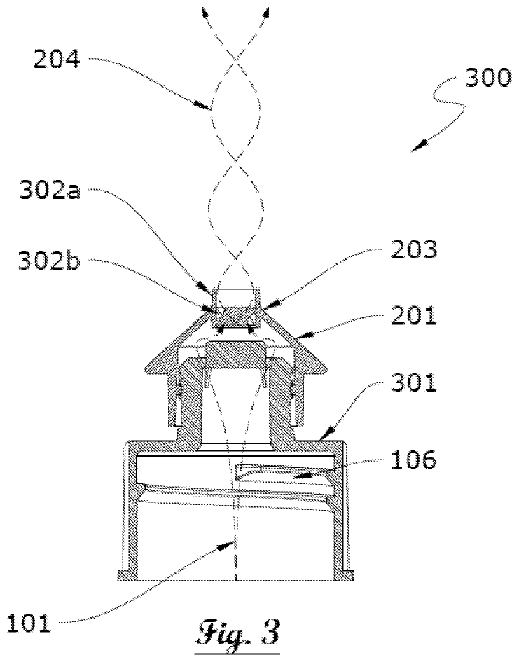

Turning now to FIG. 3, a cross sectional view a spray head 300 as described herein is shown. In some embodiments, a fluid 101 is urged to interact with the spray head 300 by way of modulating the pressure acting on the fluid. As fluid enters the body 301 of the spray head, it may interact with a turbulating structure 203. In some arrangements, such a turbulating structure comprises a plurality of canted holes 302b being disposed at an angle relative to the forwardmost surface 104. Not wishing to be limited by any particular theory, water that enters the canted holes 302b experiences a change in momentum, often by gaining angular momentum in conjunction with upwardly extending linear momentum. A fence structure 302a is mounted superjacent said plurality of holes 302b, the fence is configured to apply a normal force to counter the centrifugal force of the whirling vortex of fluid.

In another aspect, the disclosure comprises a spray head having a sealing means 106 adapted for securing the cap to a bottle. In some embodiments, a plurality of threads configured to interact with threads on a bottle are preferred.

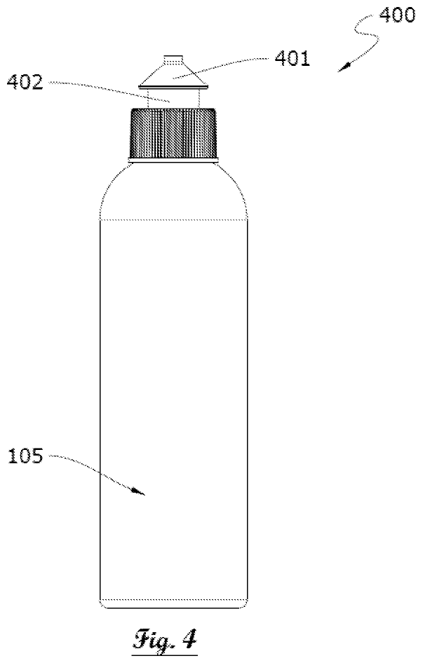

Turning now to FIG. 4, a frontal view of a bottle and in some exemplary embodiments, the nozzle assembly 400 is given. In such an arrangement, it is envisioned that a user may manipulate pressure onto the bottle assembly causing the fluid therein to become pressurized. While such pressurization occurs, the fluid may be encouraged to enter the spray head and interact with the turbulating assembly 401 housed within the cap 402. The turbulating assembly is configured to transform the momentum of the fluid into a vector having an angular and linear component. Such transformation of momentum establishes increased turbulent, and in some cases, increased rinsing.

### List of Embodiments

The following is a non-limiting list of embodiments:

Embodiment 1. A device for delivering fluid to a surface (e.g., body cavity, body surface, plastic surface, metal surface etc.) and rinse thereof, said device comprising:
a housing having a forwardmost end and a trailing end, the trailing end formed to be received by a container which may be removable and be in fluid communication therewith;
a hollow cavity defining the interior of said housing;
and an interface disposed on the forwardmost end of said housing to be received by a surface;
wherein said forwardmost end comprises a turbulating structure characterized by a plurality of holes having an orientation at an angle from square to said interface, and a fence perpendicular to said interface superjacent said turbulating structure.

Embodiment 2. The device of Embodiment 1, wherein the surface comprises a body cavity, body surface, plastic surface, or a metal surface.

Embodiment 3. The device of Embodiment 1, wherein said turbulating structure is configured such that pressurized fluid, when urged to interact with said plurality of holes and said fence, gains angular momentum in conjunction with upward extending linear momentum forms a vortex and experiences increased turbulence.

Embodiment 4. The device of Embodiment 1, wherein said fence is an elongated member having an annular cross section configured to apply a normal force to the centrifugal force of the fluid flow.

Embodiment 5. The device of Embodiment 1, further comprising a deformably resilient fluid container, having an opening at one side thereof, for containing fluid therein.

Embodiment 6. The device of Embodiment 1, further comprising a linear elongated member defined by an annular interior disposed on the trailing end of said housing to be in fluid communication with the fluid container.

Embodiment 7. The device of Embodiment 1, wherein said plurality of holes comprises at least 1 or more holes and wherein said holes are situated in a pattern.

Embodiment 8. The device of Embodiment 1, wherein said fence is defined by a ring or annular tube and whose length is configured to optimize the exiting fluid flow.

Embodiment 9. The device of Embodiment 1, wherein said angle is between about 10 and 80 degrees relative to the front of said interface.

Embodiment 10. The device of Embodiment 1, wherein said angle is between about 20 and 30 degrees relative to the front of said interface.

Embodiment 11. The device of Embodiment 1, wherein said angle is about 26 degrees relative to said interface.

Embodiment 12. The device of Embodiment 1, wherein said interface comprises a structure in which a forward side protrudes to encounter the surface such as a wide heart form, a hemisphere form, a circular cone form, or a circular truncated cone form.

Embodiment 13. The device of Embodiment 1, wherein said adapter is detachably mountable to a deformably resilient fluid container.

Embodiment 14. The device of Embodiment 5, wherein the deformable resilient fluid container is a plastic container.

Embodiment 16. The device of Embodiment 2, wherein said body cavity is at least one selected from but not limited to the group: nasal cavity, paranasal sinuses, external auditory canal, nasopharynx, eustachian tube, oral cavity, oropharynx, hypopharynx, larynx, trachea, lung, esophagus, stomach, anus, rectum, large and small intestine, urethra, bladder, vagina, cervix, uterus, eyes, and eye socket.

Embodiment 17. The device of Embodiment 1, wherein said device is configured for a dosage delivery of a fluid.

Embodiment 18. A method for applying a fluid to a surface (e.g., a body cavity, body surface, plastic surface, metal surface etc.), the method comprising using the apparatus of Embodiment 1 to apply said fluid.

Embodiment 19. The method of Embodiment 18, wherein said fluid is applied by;
a. sealingly engaging said interface with a body cavity
b. pressurizing a fluid urging the fluid to enter, and while so entered, engaging with the apparatus;
c. a fluid injection step in which a turbulent vortex of fluid generated by said pressurized fluid engaging said apparatus is injected into a body cavity;
d. a rinsing step in which the body cavity is rinsed upon interaction with said vortex of fluid.

Embodiment 20. The method of Embodiment 19, wherein the body cavity wherein said cavity is at least one selected from but not limited to the group: nasal cavity, paranasal sinuses, external auditory canal, nasopharynx, eustachian tube, oral cavity, oropharynx, hypopharynx, larynx, trachea, lung, esophagus, stomach, anus, rectum, large and small intestine, urethra, bladder, vagina, cervix, uterus, eyes, and eye socket.

Embodiment 21. The method of Embodiment 19 wherein said rinsing step further comprises rinsing from the turbulence of sloshing in the body cavity upon interaction with said vortex of fluid.

Embodiment 22. The method of Embodiment 19, wherein said turbulent fluid is applied for treating at least one of rhinitis, sinusitis, common cold, influenza, thick nasal mucus, allergens, nasal sinus polyps, and nasal obstruction.

Embodiment 23. The method of Embodiment 18, wherein the fluid is medicated and applied for the treating of a viral, fungal, bacterial infection as well as polyps or inflammation.

Embodiment 24. The method of Embodiment 23, wherein the viral infection is caused by at least one selected from: rhinovirus, coronavirus, influenza virus, adenovirus, human parainfluenza virus, respiratory syncytial virus, enterovirus other than rhinovirus, and metapneumovirus and others.

Embodiment 25. The method of Embodiment 23, wherein the bacterial infection is caused by at least one selected from: Streptococcus pneumoniae, Haemophiles influenzae, Moraxella catarrhalis, Staphylococcus aureus, and other streptococci strains that are pathogenic bacteria causative of sinusitis, anaerobic bacteria, Pseudomonas aeruginosa and Gram-negative bacteria and others.

Embodiment 26. The method of Embodiment 23, wherein the fungal infection is caused by at least one selected from: Rhizopus, Mucor, Cladosporium, Candida, Curvularia lunata, Aspergillus fumigatus, Bipolaris and Drechslera and others.

Embodiment 27. The method of Embodiment 18, wherein said fluid is water.

Embodiment 28. The method of Embodiment 18, wherein said fluid is saline.

Embodiment 29. The method of Embodiment 18, wherein said fluid is comprises one or more medications.

Embodiment 30. A nozzle assembly comprising:
a housing of the type having a body that has an inlet thereto and an outlet therefrom;
a core member and a nozzle structure, said core member defining the interior of said nozzle and providing fluid communication between the inlet and the outlet; said nozzle structure having a plurality of holes about the outlet of said nozzle and a fence structure perpendicular to said holes;
   wherein said holes of said nozzle have a canted orientation relative to a forwardmost surface.

Embodiment 31. The nozzle assembly of Embodiment 30, further comprising a tubular member situated proximally on said inlet in fluid communication with a container.

Embodiment 32. The nozzle assembly of Embodiment 30, wherein said canted orientation is between 10 and 80 degrees relative to the outlet.

Embodiment 33. The nozzle assembly of Embodiment 30, wherein said canted orientation is between 20 and 30 degrees relative to the outlet.

Embodiment 34. The nozzle assembly of Embodiment 30, wherein said canted orientation is about 26 deg. relative to the outlet.

Embodiment 35. The nozzle assembly of Embodiment 30, wherein said plurality of holes and said fence is configured such that fluid urged from said nozzle into interaction therewith experiences increased turbulence thus forming a vortex.

Embodiment 36. The nozzle assembly of Embodiment 30, wherein the inlet is fixably secured a deformably resilient fluid container.

Embodiment 37. The nozzle assembly of Embodiment 36, wherein the inlet is fixably secured to said deformably resilient fluid container via a press fit connection.

Embodiment 38. The nozzle assembly of Embodiment 36, wherein the inlet is fixably secured to said deformably resilient fluid container via a threaded connection.

Embodiment 39. The nozzle assembly of Embodiment 30, wherein the outlet comprises a spray head.

Embodiment 40. The nozzle assembly of Embodiment 30, further comprising a substantially fluid resistant seal therebetween said interface and said container.

Embodiment 41. The nozzle assembly of Embodiment 30, further comprising means for sealing the nozzle and discontinuing fluid communication.

Embodiment 42. A device, comprising:
a housing having an orifice that opens into a chamber; and
a cap sealingly attached thereto;
wherein the cap has a first end with a first opening, a second end with chamfered tip having a plurality of second openings disposed in an annular arrangement and having a fence for directing fluid flow, and a hollow channel extending from the first opening to the plurality of second openings;
wherein the first end is attached to the housing proximate the orifice;
wherein the tip is shaped and dimensioned to be inserted partially into, and form a substantially fluid-resistant seal with, an external orifice;
wherein the tip defines a flow path having a longitudinal axis;
wherein each of the plurality of second openings is canted relative to the longitudinal axis toward an adjacent second opening such that, as a liquid is first urged simultaneously through the plurality of second openings and second interacts with said fence, the liquid forms a vortex flow pattern.

1. A device for delivering fluid to a surface said device comprising:
a housing having a forwardmost end and a trailing end, the trailing end formed to be received by a container and be in fluid communication therewith;
a hollow cavity defining the interior of said housing;
and an interface disposed on the forwardmost end of said housing to be received by a surface;
wherein said forwardmost end comprises a turbulating structure characterized by a plurality of holes having an orientation at an angle from square to said interface, and a fence perpendicular to said interface superjacent said turbulating structure.

2. The device of claim 1, wherein said turbulating structure is configured such that pressurized fluid, when urged to interact with said plurality of holes and said fence, gains angular momentum in conjunction with upward extending linear momentum forms a vortex and experiences increased turbulence.

3. The device of claim 1, wherein said fence is an elongated member having an annular cross section configured to apply a normal force to the centrifugal force of the fluid flow.

4. The device of claim 1, further comprising a deformably resilient fluid container, having an opening at one side thereof, for containing fluid therein.

5. (canceled)

6. The device of claim 1, further comprising a linear elongated member defined by an annular interior disposed on the trailing end of said housing to be in fluid communication with the fluid container.

7. (canceled)

8. (canceled)

9. The device of claim 1, wherein said angle is between about 10 and 80 degrees relative to the front of said interface.

10. (canceled)

11. (canceled)

12. The device of claim 1, wherein said interface comprises a structure in which a forward side protrudes to encounter the surface such as a wide heart form, a hemisphere form, a circular cone form, or a circular truncated cone form.

13. The device of claim 1, wherein said adapter is detachably mountable to a deformably resilient fluid container.

14. (canceled)

15. (canceled)

16. The device of claim 1, wherein said device is configured for a dosage delivery of a fluid.

17. A method for applying a fluid to a surface, said method comprising using an apparatus to apply said fluid, said apparatus comprising:
a housing having a forwardmost end and a trailing end, the trailing end formed to be received by a container and be in fluid communication therewith;
a hollow cavity defining the interior of said housing;
and an interface disposed on the forwardmost end of said housing to be received by a surface;
wherein said forwardmost end comprises a turbulating structure characterized by a plurality of holes having an orientation at an angle from square to said interface, and a fence perpendicular to said interface superjacent said turbulating structure.

18. The method of claim 17, wherein said fluid is applied by;
a. sealingly engaging said interface with a body cavity;
b. pressurizing a fluid urging the fluid to enter, and while so entered, engaging with the apparatus;
c. a fluid injection step in which a turbulent vortex of fluid generated by said pressurized fluid engaging said apparatus is injected into a body cavity;
d. a rinsing step in which the body cavity is rinsed upon interaction with said vortex of fluid.

19. The method of claim 18, wherein the body cavity wherein said cavity is at least one selected from but not limited to the group: nasal cavity, paranasal sinuses, external auditory canal, nasopharynx, eustachian tube, oral cavity, oropharynx, hypopharynx, larynx, trachea, lung, esophagus, stomach, anus, rectum, large and small intestine, urethra, bladder, vagina, cervix, uterus, eyes, and eye socket.

20. (canceled)

21. (canceled)

22. (canceled)

23. (canceled)

24. (canceled)

25. (canceled)

26. (canceled)

27. (canceled)

28. (canceled)

29. (canceled)

30. A nozzle assembly comprising:
a housing of the type having a body that has an inlet thereto and an outlet therefrom;
a core member and a nozzle structure, said core member defining the interior of said nozzle and providing fluid communication between the inlet and the outlet; said nozzle structure having a plurality of holes about the outlet of said nozzle and a fence structure perpendicular to said holes;
wherein said holes of said nozzle have a canted orientation relative to a forwardmost surface.

31. The nozzle assembly of claim 30, further comprising a tubular member situated proximally on said inlet in fluid communication with a container.

32. The nozzle assembly of claim 30, wherein said canted orientation is between 10 and 80 degrees relative to the outlet.

33. (canceled)

34. (canceled)

35. The nozzle assembly of claim 30, wherein said plurality of holes and said fence is configured such that fluid urged from said nozzle into interaction therewith experiences increased turbulence thus forming a vortex.

36. The nozzle assembly of claim 30, wherein the inlet is fixably secured to a deformably resilient fluid container.

37. (canceled)

38. (canceled)

39. The nozzle assembly of claim 30, wherein the outlet comprises a spray head.

40. The nozzle assembly of claim 30, further comprising a substantially fluid resistant seal therebetween said interface and said container.

41. The nozzle assembly of claim 30, further comprising means for sealing the nozzle and discontinuing fluid communication.

42. (canceled)
